(19) United States
(12) Patent Application Publication
(10) Pub. No.: US 2016/0090628 A1
(43) Pub. Date: Mar. 31, 2016

(54) DETECTION OF MINERALOCORTICOID RECEPTOR ACTIVATION AND PERSONALIZED ANTIHYPERTENSIVE THERAPY BASED THEREON

(71) Applicant: The Regents of University of Michigan, Ann Arbor, MI (US)

(72) Inventors: James Brian Byrd, Ann Arbor, MI (US); Richard J. Auchus, Ann Arbor, MI (US); William Rainey, Ann Arbor, MI (US)

(21) Appl. No.: 14/865,901

(22) Filed: Sep. 25, 2015

Related U.S. Application Data
(60) Provisional application No. 62/055,385, filed on Sep. 25, 2014.

(51) Int. Cl. C12Q 1/68

(52) U.S. Cl. CPC C12Q 1/6883; C12Q 2600/158; C12Q 2600/106

(57) ABSTRACT

Provided herein are compositions and methods for the assessment of mineralocorticoid receptor activation or repression, and methods of customizing antihypertensive therapies based thereon. In particular, assays are provided for the detection of targets of mineralocorticoid receptor activation.

### CROSS-REFERENCE TO RELATED APPLICATION

The present application claims priority to U.S. Provisional Patent Application Ser. No. 62/055,385 filed Sep. 25, 2014, which is hereby incorporated by reference in its entirety.

### FIELD

Provided herein are compositions and methods for the assessment of mineralocorticoid receptor activation or repression, and methods of customizing antihypertensive therapies based thereon. In particular, assays are provided for the detection of targets of mineralocorticoid receptor activation.

### BACKGROUND

Hypertension is prevalent, affecting 33% of the United States' adult population. Effective treatment of hypertension reduces the risk of death, myocardial infarction, heart failure, and stroke. However, hypertension is commonly resistant to treatment. Approximately 20% of patients with resistant hypertension have primary aldosteronism, in which inappropriate aldosterone secretion leads to activation of the mineralocorticoid receptor (MR). Activation of the MR increases the expression of the amiloride-sensitive epithelial sodium channel (ENaC) in the distal nephron, resulting in sodium and water retention plus loss of potassium. MR antagonists (MRAs) are effective in the treatment of primary aldosteronism. Beyond primary aldosteronism, MRAs are also an effective antihypertensive treatment in many patients with normal or low circulating levels of aldosterone. For example, in resistant hypertension, MR activation is increased, as evidenced by a robust response to MRA therapy. In the randomized placebo-controlled ASPIRANT trial, the MRA spironolactone reduced 24-hour systolic blood pressure in resistant hypertension by a mean of 9.8 mm Hg more than did placebo. Other studies have shown 16-26 mm Hg mean reductions in systolic blood pressure after addition of spironolactone in resistant hypertension. Serum aldosterone, however, does not predict response to spironolactone, suggesting illicit activation of MR by an unknown non-aldosterone ligand. Because it is not possible to predict which patients' blood pressure will respond well to MRAs, an uncertain risk-benefit ratio has precluded the common use of MRAs in hypertension.

One approach to this problem is to measure urinary electrolytes, but urinary sodium and potassium vary according to dietary intake and medications. Therefore, inferences regarding the activation of MR based upon urinary sodium and potassium are only reasonable if the diet is carefully controlled prior to testing. This limitation renders this method of assessment impractical. The aldosterone/renin ratio (ARR) helps to identify cases of primary and secondary aldosteronism, but many medications confound interpretation of this ratio. More importantly, spironolactone-responsive hypertension often occurs in the setting of a normal or low aldosterone level and normal ARR. Thus, MR activation can occur when the serum aldosterone concentration is not elevated.

The immediate downstream effect of MR activation is increased expression of heteromultimeric amiloride-sensitive epithelial sodium channels (ENaC) on the luminal surface of epithelial cells in the distal nephron. Aldosterone binds to MR in epithelial cells of the distal nephron. The aldosterone-MR complex translocates to the nucleus, where it acts as a transcription factor for the α, β, and γ subunits of ENaC, as well as other genes. ENaC is trafficked to the luminal surface of the membrane, where it enhances sodium and water reabsorption and promotes potassium excretion. The aldosterone-MR complex also activates genes that prevent internalization and degradation of membrane-bound ENaC. Prior approaches to measuring ENaC subunits would be difficult or impossible to apply in a clinical setting or have yielded difficult-to-explain results. Prior assays of human urinary ENaC subunits have involved the isolation of urinary exosomes, a component of urine isolated through prolonged and often multiple episodes of ultracentrifugation. If reliable results can be obtained without ultracentrifugation, translation to the clinic becomes much easier since clinical labs do not typically have ultracentrifuges.

### SUMMARY

Provided herein are compositions and methods for the assessment of mineralocorticoid receptor activation or repression, and methods of customizing antihypertensive therapies based thereon. In particular, assays are provided for the detection of targets (e.g., protein, mRNA, etc.) of mineralocorticoid receptor activation (e.g., epithelial sodium channel (ENaC), GILZ, etc.). Methods and compositions described herein find use in a variety of clinical applications, including, but not limited to: identifying hypertensive patients likely to benefit from MRA therapy, assessing the completeness of MR blockade in states of aldosterone excess (e.g., heart failure and primary aldosteronism), and diagnosing primary aldosteronism. More broadly, a biomarker of MR activation would be an important tool for investigating the pathophysiology of low-renin hypertension in patients with normal circulating aldosterone, as is commonly observed in obesity-associated hypertension.

In some embodiments, provided herein are methods for detecting or quantifying one or more target analytes that are indicative of mineralocorticoid receptor activation in a subject, the method comprising exposing a urine sample from the subject to detection reagents that are specific for the target analytes, wherein the urine sample has not been subjected to ultracentrifugation. In some embodiments, the target analytes are selected from SCNN1A (encoding ENaC α), SCNN1B (encoding ENaC β), SCNN1G (encoding ENaC γ), TSC22D3 (encoding GILZ), SGK1, PER1, FKBP5, RASL12, SLC12A3, TNS1, and KCNJ1. In some embodiments, the target analytes are selected from Akap12, Ophn1, Apbb3, Per1, Asap1, Cp, Ctgf, Slc45a1, Fgd3, Slco3a1, Synpo, Ikzf4, Tgfa, Klf6, Klf9, Mrpl33, Tspan2, Msi2, Zfand5, and Ngf. In some embodiments, the urine sample is processed (e.g., filtered, centrifuged at sub-ultracentrifugation speeds, concentrated, diluted, etc.), but not ultracentrifuged. In some embodiments, the target analytes are mRNA transcripts of genes expressed following mineralocorticoid receptor activation, or nucleic acid fragments thereof. In some embodiments, the detection reagents comprise detectably-labeled nucleic acid probes that specifically hybridize to the target analytes or amplification products thereof. In some embodiments, the detection reagents are fluorescently labeled. In some embodiments, detection reagents are selected from (i) non-specific fluorescent dyes that intercalate amplification products of target analytes, and (ii) fluorescently-labeled and target-specific oligonucleotide probes. In some embodiments, methods further comprise exposing a urine sample from the subject to amplification reagents that are specific for the target analytes. In some embodiments, the amplification reagents comprise target-analyte-specific primers. In some embodiments, the urine sample is exposed to two or more pairs of target-analyte-specific primers for each target analyte. In some embodiments, the urine sample is contacted with a detection reagent for each of the two or more pairs of target-analyte-specific primers. In some embodiments, methods comprise the steps of (a) obtaining or receiving the urine sample; (b) processing the urine sample; (c) amplifying portions of one or more of the target analytes using target-analyte-specific primers pairs to produce one or more target-analyte-specific amplicons; (d) contacting the urine sample with at least one detection probe for each of the one or more target-analyte-specific amplicons.

In some embodiments, provided herein are methods for quantitative or semiquantitative detection of downstream targets (e.g., protein, mRNA, etc.) of mineralocorticoid receptor (MR) activation including, but not limited to SCNN1A (encoding ENaC α), SCNN1B (encoding ENaC β), SCNN1G (encoding ENaC γ), TSC22D3 (encoding GILZ), SGK1, PER1, FKBP5, RASL12, SLC12A3, TNS1, KCNJ1, etc.). In some embodiments, provided herein are methods for quantitative or semiquantitative detection of other downstream targets (e.g., protein, mRNA, etc.) of mineralocorticoid receptor (MR) activation, including, but not limited to Akap12, Ophn1, Apbb3, Per1, Asap1, Cp, Ctgf, Slc45a1, Fgd3, Slco3a1, Synpo, Ikzf4, Tgfa, Klf6, Klf9, Mrpl33, Tspan2, Msi2, Zfand5, Ngf, etc. In some embodiments, the downstream targets of MR are biomarkers of MR activation. In other embodiments, biomarkers of MR-repression are detected.

In some embodiments, MR-activation biomarkers are detected and/or quantitated in a biological sample obtained from a subject. A subject may be a human, non-human primate, mouse, rat, or other mammalian subject. In some embodiments, a biological sample comprises urine or a urine product (e.g., urinary exosomes, salt-depleted urine, untreated urine, concentrated urine, diluted urine, etc.), blood or a blood product (e.g., serum, plasma, or whole blood, exosomes isolated from blood), tears, or other body fluids or tissues (e.g., from a human subject). A sample may be processed (e.g., concentrated, diluted, salt-depleted, precipitated, lysed, extracted, centrifuged, denatured, etc.) or unprocessed.

In some embodiments, a sample (e.g., urine sample) is not ultracentrifuged. In some embodiments, a samples is not exposed to centrifugation at speeds in excess of 20,000 rpm, 25,000 rpm, 30,000 rpm, 40,000 rpm, 50,000 rpm, 60,000 rpm, 70,000 rpm, or values and ranges therein. In some embodiments, a sample (e.g., urine or blood product) is centrifuged at sub-ultracentrifugation speeds (e.g., <20,000 rpm, 18,000 rpm, 16,000 rpm, 14,000 rpm, 12,000 rpm, 10,000 rpm, 8,000 rpm, 6,000 rpm, 4,000 rpm, 2,000 rpm, and values and ranges therein) prior to further analysis (e.g., detection and/or quantification of biomarkers). In some embodiments, a sample is not exposed to relative centrifugal forces in excess of 20,000 g, 30,000 g, 40,000 g, 50,000 g, 60,000 g, 70,000 g, 80,000 g, 90,000 g, or 100,000 g (e.g., ultracentrifugal forces). In some embodiments, a sample is exposed to relative centrifugal forces below 100,000 g, 90,000 g, 80,000 g, 70,000 g, 60,000 g, 50,000 g, 40,000 g, 30,000 g, 20,000 g, or 10,000 g (e.g., conventional centrifugal forces, sub-ultracentrifugal forces, etc.). In some embodiments, the sample (e.g., urine or blood product) is not centrifuged. In some embodiments, the sample (e.g., urine or blood product) is not subjected to protease inhibitors (or cocktails thereof).

In some embodiments, MR-activation or MR-repression biomarkers are proteins or protein subunits (e.g., ENaC α, ENaC β, ENaC γ, GILZ, SGK1, PER1, FKBP5, RASL12, SLC12A3, TNS1, KCNJ1, etc.) that are targets of MR or downstream products of MR activation or repression. In some embodiments, detection and/or quantification reagents are provided. In embodiments in which a biomarker is a protein, polypeptide and/or peptide, detection and/or quantification reagents may comprise antibodies or antibody-like reagents, aptamers, etc. that bind (e.g., specifically) to the specific MR-activation or MR-repression biomarkers. In such embodiments, detection and/or quantification may be achieved by, for example, an immunoassay, Western blot, enzyme-linked immunosorbent assay (ELISA), radioimmunoassay (RIA), fluorimetric assay, or other suitable assays known in the field.

In some embodiments, MR-activation or MR-repression biomarkers are RNAs (e.g., mRNA) encoding proteins or subunits thereof (e.g., ENaC α, ENaC β, ENaC γ, GILZ, SGK1, PER1, FKBP5, RASL12, SLC12A3, TNS1, KCNJ1, etc.) that are targets of MR or downstream products of MR activation or repression. In embodiments in which a biomarker is an RNA (e.g., mRNA), detection and/or quantification reagents may comprise primers (e.g., for amplification, reverse transcription, etc.) or probes (e.g., detectably-labeled (e.g., optically-labeled, fluorescently labeled, etc.) oligonucleotides) that bind (e.g., specifically) to the MR-activation or MR-repression biomarker. In such embodiments, detection and/or quantification may be achieved by, for example, RT-PCR, qPCR, Northern blot analysis, an enzymatic cleavage assay (e.g., INVADER, Hologic, Inc.; See e.g., U.S. Pat. Nos. 5,846,717, 6,090,543; 6,001,567; 5,985,557; and 5,994,069; each of which is herein incorporated by reference), a hybridization assay (e.g., TaqMan assay (Life Technologies; See e.g., U.S. Pat. Nos. 5,962,233 and 5,538,848, each of which is herein incorporated by reference), etc.

In some embodiments, reverse-transcriptase PCR(RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

In some embodiments, quantitative PCR (qPCR) or real time PCR (RT-PCR) is used to detect/quantify analytes. In some embodiments, mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR produces an absolute measurement such as number of copies of mRNA in a sample or portion of a sample.

In some embodiments, nucleic acid from a sample is sequenced (e.g., in order to detect biomarkers). Nucleic acid molecules may be sequence analyzed by any number of techniques. The analysis may identify the sequence of all or a part of a nucleic acid. Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack, experimentally RNA is usually, although not necessarily, reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties) and the Adessi PCR colony technology (Adessi et al. (2000). Nucleic Acid Res. 28, E87; WO 00018957; herein incorporated by reference in its entirety).

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, Pacific Biosciences (PAC BIO RS II) and other platforms commercialized.

In some embodiments, provided herein are methods of determining a treatment course of action for a subject suffering from hypertension or heart failure comprising: (a) quantitatively or semi-quantitatively determining an amount of: (i) one or more protein biomarkers (e.g., ENaC α, ENaC β, ENaC γ, GILZ, SGK1, PER1, FKBP5, RASL12, SLC12A3, TNS1, or KCNJ1), or (ii) mRNA encoding one or more of said protein biomarkers in a biological sample (e.g., urine or blood product) from said subject using the methods described herein, and (b) identifying said subject as: (i) responsive to treatment with mineralocorticoid receptor (MR) antagonists based on the amount determined in step (a) being above a first threshold level, or (ii) resistant to treatment with MR antagonists based on the amount determined in step (a) being below a second threshold level. In some embodiments, identifying said subject as resistant to treatment with MR antagonists indicates not prescribing MR antagonists to said subject for treatment of hypertension. In some embodiments, identifying said subject as responsive to treatment with MR antagonists indicates prescribing MR antagonists to said subject for treatment of hypertension. In some embodiments, methods further comprise prescribing a treatment of hypertension (e.g., MR antagonist, other treatment).

In some embodiments, the provided herein are methods comprising: (a) identifying a subject as suffering from mineralocorticoid hypertension (e.g., using an assay for mineralocorticoid hypertension); (b) identifying said subject as: (i) resistant to treatment with mineralocorticoid receptor (MR) antagonists based on an amount of: (i) one or more protein biomarkers (e.g., ENaC α, ENaC β, ENaC γ, GILZ, SGK1, PER1, FKBP5, RASL12, SLC12A3, TNS1, or KCNJ1), or (ii) mRNA encoding one or more of said protein biomarkers in a biological sample (e.g., urine or blood product) from said subject that is outside a range for subject responsive to treatment with MR antagonists; and (c) administering an appropriate treatment for hypertension based on the findings in step (b). In some embodiments, methods further comprise step (d) of retesting said subject for hypertension and/or for responsiveness to MR antagonists.

In some embodiments, the provided herein are methods comprising: (a) identifying a subject as suffering from mineralocorticoid hypertension (e.g., using an assay for mineralocorticoid hypertension); (b) identifying said subject as: (i) responsive to treatment with mineralocorticoid receptor (MR) antagonists based on an amount of: (i) one or more protein biomarkers (e.g., ENaC α, ENaC β, ENaC γ, GILZ, SGK1, PER1, FKBP5, RASL12, SLC12A3, TNS1, or KCNJ1), or (ii) mRNA encoding one or more of said protein biomarkers in a biological sample (e.g., urine or blood product) from said subject that is outside a range for subject resistant to treatment with MR antagonists; and (c) administering an appropriate treatment for hypertension based on the findings in step (b). In some embodiments, methods further comprise step (d) of retesting said subject for hypertension and/or for responsiveness to MR antagonists.

In some embodiments, the provided herein are methods of treating a subject for hypertension comprising: (a) identifying said subject as responsive or resistant to treatment with MR antagonists (e.g., by the methods described herein); (b) providing said subject with a therapy consistent with the determination of step (a). In some embodiments, the subject is resistant to treatment with MR antagonists and said subject is provided with a non-MR antagonist therapy. In some embodiments, the non-MR antagonist therapy is selected from one or more of thiazide or thiazide-like diuretic, calcium channel blockers, angiotensin converting enzyme inhibitors, beta-blockers, and alpha-blockers. In some embodiments, the subject is responsive to treatment with MR antagonists and said subject is provided with an MR antagonist therapy. In some embodiments, the MR antagonist therapy is selected from one or more of spironolactone and eplerenone.

In some embodiments, provided herein are methods of treating a subject with hypertension and/or aldosteronism comprising (a) having a sample (e.g., urine sample, blood sample, products thereof) from the subject tested for the level of: (i) one or more protein biomarkers (e.g., ENaC α, ENaC β, ENaC γ, GILZ, SGK1, PER1, FKBP5, RASL12, SLC12A3, TNS1, or KCNJ1), or (ii) mRNA encoding one or more of said protein biomarkers; (b) identifying said subject as: (i) resistant to treatment with mineralocorticoid receptor (MR) antagonists based on an amount of: (A) one or more of said protein biomarkers or (B) mRNA encoding one or more of said protein biomarkers in said sample outside of a range that indicates responsiveness to treatment, or (ii) responsive to treatment with MR antagonists based on an amount of: (A) one or more of said protein biomarkers or (B) mRNA encoding one or more of said protein biomarkers in said sample within a range that indicates responsiveness to treatment; and (c) administering or prescribing an appropriate treatment based on the findings in step (b). In some embodiments, the appropriate treatment is a mineralocorticoid receptor antagonist. In some embodiments, methods further comprise a step (d) of retesting said subject for hypertension and/or for responsiveness to MR antagonists. In some embodiments, the amount of one or more biomarkers is above or below a threshold value.

In some embodiments, provided herein are compositions comprising detection and/or capture reagents that specifically bind to a MR-activation or MR-repression biomarker. In some embodiments, the biomarker is a MR target protein (e.g., ENaC α, ENaC β, ENaC γ, GILZ, SGK1, PER1, FKBP5, RASL12, SLC12A3, TNS1, or KCNJ1) or a subunit thereof. In some embodiments, the biomarker is the complete ENaC protein, a portion of ENaC, and/or an ENaC subunit (e.g., α-subunit, β-subunit, γ-subunit, etc.). In some embodiments, a detection and/or capture reagent is an antibody, antibody-like molecule or complex, an aptamer, etc. (e.g., specific for ENaC α, ENaC 13, ENaC γ, GILZ, SGK1, PER1, FKBP5, RASL12, SLC12A3, TNS1, or KCNJ1). In some embodiments, the antibody is one used in the experiments conducted during development of embodiments described herein.

In some embodiments, provided herein are compositions comprising detection and/or capture reagents that specifically bind to a nucleic acid MR-activation or MR-repression biomarker. In some embodiments, the biomarker is an RNA (e.g., mRNA) encoding a MR target protein (e.g., ENaC α, ENaC β, ENaC γ, GILZ, SGK1, PER1, FKBP5, RASL12, SLC12A3, TNS1, or KCNJ1) or a subunit thereof. In some embodiments, the biomarker is an RNA encoding the complete ENaC protein, a portion of ENaC, and/or an ENaC subunit (e.g., α-subunit, β-subunit, γ-subunit, etc.). In some embodiments, a detection and/or capture reagent is an oligonucleotide probe comprising a portion that is complementary to encoding a MR target protein (e.g., ENaC α, ENaC β, ENaC γ, GILZ, SGK1, PER1, FKBP5, RASL12, SLC12A3, TNS1, or KCNJ1, etc.). For example, provided herein are nucleic acid oligonucleitodes comprising a portion with at least 70% sequence identity (e.g., 70%, 75%, 80%, 85%, 90%, 91%, 92%, 93%, 94%, 95%, 96%, 97%, 98%, 99%, 100%, or ranges therein) with one of ENaC α, ENaC β, ENaC γ, GILZ, SGK1, PER1, FKBP5, RASL12, SLC12A3, TNS1, or KCNJ1 or a portion thereof (e.g., 8 nt, 10 nt, 12 nt, 15 nt, 18 nt, 20 nt, 25 nt, 30 nt, 35 nt, 40 nt, 50 nt, 75 nt, 100 nt, or more, or ranges therein. In some embodiments, oligonucleotides are primers for amplifying a portion of a target RNA or DNA sequence. In some embodiments, oligonucleotides are probes (e.g., detectably labeled (e.g., fluorescently labeled), etc.) for detecting/quantifying all or a portion of a target RNA or DNA sequence.

In some embodiments, the composition further comprises human urine. In some embodiments, the human urine has been subjected to centrifugation at sub-ultracentrifugation speeds (e.g., <50,000 rpm, <40,000 rpm, <30,000 rpm, <20,000 rpm, <10,000 rpm, <5,000 rpm, <4,000 rpm, <3,000 rpm, <2,000 rpm, <1,000 rpm, etc.) and/or g-force (e.g., <100,000×g, <90,000×g, <80,000×g, <70,000×g, <60,000×g, <50,000×g, <40,000×g, <30,000×g, <20,000×g, <10,000×g, <5,000×g, <1,000×g, etc.). In some embodiments, the human urine has been subjected to centrifugation for 2 hours or less, 1 hour or less, 30 minutes or less, 20 minutes or less, 15 minutes or less, 10 minutes or less, 5 minutes or less, etc. In some embodiments, the human urine has not been subjected to centrifugation.

In some embodiments, provided herein are methods for detecting mineralocorticoid receptor (MR) activation in a subject comprising exposing urine of a human subject to detection reagents (e.g., antibodies, aptamers, etc.) for one or more of ENaC α, ENaC β, ENaC γ, GILZ, SGK1, PER1, FKBP5, RASL12, SLC12A3, TNS1, and KCNJ1, with subsequent semiquantification or quantification using a suitable assay not limited to immunoblotting, enzyme-linked immunosorbent assay, or fluorescent immunoassay. In some embodiments, methods further comprise immunoblotting, enzyme-linked immunosorbent assay, or fluorescent immunoassay of said urine of a human subject with said detection reagents (e.g., antibodies, aptamers, etc.) for one or more of ENaC α, ENaC β, ENaC γ, GILZ, SGK1, PER1, FKBP5, RASL12, SLC12A3, TNS1, and KCNJ1. In some embodiments, urine (e.g., urine from a human subject) is centrifuged at sub-ultracentrifugation speeds prior to exposure to said detection reagents. In some embodiments, the detection reagentsare those used in the experiments conducted during development of embodiments described herein.

In some embodiments, provided herein are methods for detecting mineralocorticoid receptor (MR) activation in a subject comprising exposing urine of a human subject to primers specific for ENaC subunit mRNAs with subsequent semiquantification or quantification using RT-PCR (e.g., quantitative PCR). In some embodiments, urine (e.g., urine from a human subject) is centrifuged at sub-ultracentrifugation speeds prior to exposure to said antibody for ENaC.

In some embodiments, detection of biomarkers (e.g., one or more of ENaC α, ENaC β, ENaC γ, GILZ, SGK1, PER1, FKBP5, RASL12, SLC12A3, TNS1, and KCNJ1ENaC) in a body fluid or tissue (e.g., blood, urine, etc.) is performed with one or more additional assays (e.g., exosome isolation). In some embodiments, biomarker (e.g., ENaC α, ENaC β, ENaC γ, GILZ, SGK1, PER1, FKBP5, RASL12, SLC12A3, TNS1, and KCNJ1ENaC) protein or mRNA is on a panel of biomarkers (e.g., urine biomarkers, blood biomarkers, etc.) tested for determining responsiveness to treatment (e.g., for hypertension). In some embodiments, provided herein are panels of two or more markers (e.g., one or more of ENaC α, ENaC β, ENaC γ, GILZ, SGK1, PER1, FKBP5, RASL12, SLC12A3, TNS1, and KCNJ1ENaC and 1 additional marker, 2 additional markers, 5 additional markers, 10 additional markers, 20 additional markers, or more). In some embodiments, the one or more of ENaC α, ENaC β, ENaC γ, GILZ, SGK1, PER1, FKBP5, RASL12, SLC12A3, TNS1, and KCNJ1ENaC is on a panel of urine biomarkers for identifying a variety of conditions (e.g., hypertension-related or non-hypertension related). In some embodiments, the biomarkers are tested for determining the completeness of response to MR antagonist treatment. In some embodiments, the biomarkers are tested for diagnosing primary or secondary aldosteronism.

In some embodiments, provided herein are methods of determining a treatment course of action for a subject suffering from hypertension or heart failure comprising: (a) quantitatively or semi-quantitatively determining an amount of: (i) ENaC protein, (ii) one or more ENaC subunits (e.g., α-, β-, and/or γ-subunit proteins), or (iii) mRNA encoding ENaC protein or one or more ENaC subunits (e.g., α-, β-, and/or γ-subunits) in a biological sample (e.g., urine or blood product) from said subject using the methods described herein, and (b) identifying said subject as: (i) responsive to treatment with mineralocorticoid receptor (MR) antagonists based on the amount determined in step (a) being above a first threshold level, or (ii) resistant to treatment with MR antagonists based on the amount determined in step (a) being below a second threshold level. In some embodiments, identifying said subject as resistant to treatment with MR antagonists indicates not prescribing MR antagonists to said subject for treatment of hypertension. In some embodiments, identifying said subject as responsive to treatment with MR antagonists indicates prescribing MR antagonists to said subject for treatment of hypertension. In some embodiments, methods further comprise prescribing a treatment of hypertension (e.g., MR antagonist, other treatment).

In some embodiments, the provided herein are methods comprising: (a) identifying a subject as suffering from mineralocorticoid hypertension (e.g., using an assay for mineralocorticoid hypertension); (b) identifying said subject as: (i) resistant to treatment with mineralocorticoid receptor (MR) antagonists based on a below-threshold amount of: (i) ENaC protein, (ii) one or more ENaC subunits (e.g., α-, β-, and/or γ-subunits), or (iii) mRNA encoding ENaC protein or one or more ENaC subunits (e.g., α-, β-, and/or γ-subunits) in a biological sample (e.g., urine or blood product) from said subject, or (ii) responsive to treatment with MR antagonists based on an above-threshold amount of: (i) ENaC protein, (ii) one or more ENaC subunits (e.g., α-, β-, and/or γ-subunits), or (iii) mRNA encoding ENaC protein or one or more ENaC subunits (e.g., α-, β-, and/or γ-subunits) in a biological sample (e.g., urine or blood product) from said subject; and (c) administering an appropriate treatment for hypertension based on the findings in step (b). In some embodiments, methods further comprise step (d) of retesting said subject for hypertension and/or for responsiveness to MR antagonists.

In some embodiments, the provided herein are methods of treating a subject for hypertension comprising: (a) identifying said subject as responsive or resistant to treatment with MR antagonists (e.g., by the methods described herein); (b) providing said subject with a therapy consistent with the determination of step (a). In some embodiments, the subject is resistant to treatment with MR antagonists and said subject is provided with a non-MR antagonist therapy. In some embodiments, the non-MR antagonist therapy is selected from one or more of thiazide or thiazide-like diuretic, calcium channel blockers, angiotensin converting enzyme inhibitors, beta-blockers, and alpha-blockers. In some embodiments, the subject is responsive to treatment with MR antagonists and said subject is provided with an MR antagonist therapy. In some embodiments, the MR antagonist therapy is selected from one or more of spironolactone and eplerenone.

In some embodiments, provided herein are methods of treating a subject with hypertension and/or aldosteronism comprising (a) having a sample (e.g., urine sample, blood sample) from the subject tested for the level of: (i) ENaC protein, (ii) one or more ENaC subunits (e.g., α-, β-, and/or γ-subunits), or (iii) mRNA encoding ENaC protein or one or more ENaC subunits (e.g., α-, β-, and/or γ-subunits); (b) identifying said subject as: (i) resistant to treatment with mineralocorticoid receptor (MR) antagonists based on a below-threshold amount of: (i) ENaC protein, (ii) one or more ENaC subunits (e.g., α-, β-, and/or γ-subunits), or (iii) mRNA encoding ENaC protein or one or more ENaC subunits (e.g., α-, β-, and/or γ-subunits) in a biological sample (e.g., urine or blood product) from said subject, or (ii) responsive to treatment with MR antagonists based on an above-threshold amount of: (i) ENaC protein, (ii) one or more ENaC subunits (e.g., α-, β-, and/or γ-subunits), or (iii) mRNA encoding ENaC protein or one or more ENaC subunits (e.g., α-, β-, and/or γ-subunits) in a biological sample (e.g., urine or blood product) from said subject; and (c) administering or prescribing an appropriate treatment based on the findings in step (b). In some embodiments, the appropriate treatment is a mineralocorticoid receptor antagonist. In some embodiments, methods further comprise a step (d) of retesting said subject for hypertension and/or for responsiveness to MR antagonists.

In some embodiments, provided herein are compositions comprising detection and/or capture reagents that specifically bind to a MR-activation or MR-repression biomarker. In some embodiments, the biomarker is a MR target protein or a subunit thereof (e.g., ENaC, GILZ, etc.). In some embodiments, the biomarker is the complete ENaC protein, a portion of ENaC, and/or an ENaC subunit (e.g., α-subunit, β-subunit, γ-subunit, etc.). In some embodiments, a detection and/or capture reagent is an antibody, antibody-like molecule or complex, an aptamer, etc. (e.g., for ENaC protein or subunit or related MR activation-regulated proteins (e.g., GILZ, etc.)). In some embodiments, the antibody is one used in the experiments conducted during development of embodiments described herein.

In some embodiments, provided herein are compositions comprising detection and/or capture reagents that specifically bind to a nucleic acid MR-activation or MR-repression biomarker. In some embodiments, the biomarker is an RNA (e.g., mRNA) encoding a MR target protein or a subunit thereof (e.g., ENaC, GILZ, etc.). In some embodiments, the biomarker is an RNA encoding the complete ENaC protein, a portion of ENaC, and/or an ENaC subunit (e.g., α-subunit, β-subunit, γ-subunit, etc.). In some embodiments, a detection and/or capture reagent is an oligonucleotide probe comprising a portion that is complementary to encoding a MR target protein or a subunit thereof (e.g., ENaC, GILZ, etc.). For example, provided herein are nucleic acid oligonucleitodes comprising a portion with at least 70% sequence identity (e.g., 70%, 75%, 80%, 85%, 90%, 91%, 92%, 93%, 94%, 95%, 96%, 97%, 98%, 99%, 100%, or ranges therein) with the complete ENaC protein, a portion of ENaC (e.g., 8 nt, 10 nt, 12 nt, 15 nt, 18 nt, 20 nt, 25 nt, 30 nt, 35 nt, 40 nt, 50 nt, 75 nt, 100 nt, or more, or ranges therein), an ENaC subunit (e.g., α-subunit, β-subunit, γ-subunit, etc.), or a portion of an ENaC subunit (e.g., 8 nt, 10 nt, 12 nt, 15 nt, 18 nt, 20 nt, 25 nt, 30 nt, 35 nt, 40 nt, 50 nt, 75 nt, 100 nt, or more, or ranges therein). In some embodiments, oligonucleotides are primers for amplifying a portion of a target RNA or DNA sequence. In some embodiments, oligonucleotides are probes (e.g., detectably labeled (e.g., fluorescently labeled), etc.) for detecting/quantifying all or a portion of a target RNA or DNA sequence.

In some embodiments, the composition further comprises human urine. In some embodiments, the human urine has been subjected to centrifugation at sub-ultracentrifugation speeds (e.g., <50,000 rpm, <40,000 rpm, <30,000 rpm, <20,000 rpm, <10,000 rpm, <5,000 rpm, <4,000 rpm, <3,000 rpm, <2,000 rpm, <1,000 rpm, etc.) and/or g-force (e.g., <100,000×g, <90,000×g, <80,000×g, <70,000×g, <60,000×g, <50,000×g, <40,000×g, <30,000×g, <20,000×g, <10,000×g, <5,000×g, <90,000×g, etc.). In some embodiments, the human urine has been subjected to centrifugation for 2 hours or less, 1 hour or less, 30 minutes or less, 20 minutes or less, 15 minutes or less, 10 minutes or less, 5 minutes or less, etc. In some embodiments, the human urine has not been subjected to centrifugation.

In some embodiments, provided herein are methods for detecting mineralocorticoid receptor (MR) activation in a subject comprising exposing urine of a human subject to an antibody for ENaC subunit protein with subsequent semiquantification or quantification using immunoblotting, enzyme-linked immunosorbent assay, or fluorescent immunoassay. In some embodiments, methods further comprise immunoblotting, enzyme-linked immunosorbent assay, or fluorescent immunoassay of said urine of a human subject with said antibody for ENaC subunit proteins. In some embodiments, urine (e.g., urine from a human subject) is centrifuged at sub-ultracentrifugation speeds prior to exposure to said antibody for ENaC subunit proteins. In some embodiments, the antibody is one used in the experiments conducted during development of embodiments described herein.

In some embodiments, provided herein are methods for detecting mineralocorticoid receptor (MR) activation in a subject comprising exposing urine of a human subject to primers specific for ENaC subunit mRNAs with subsequent semiquantification or quantification using RT-PCR. In some embodiments, urine (e.g., urine from a human subject) is centrifuged at sub-ultracentrifugation speeds prior to exposure to said antibody for ENaC.

In some embodiments, detection of ENaC subunit proteins in the urine is performed with one or more additional assays. In some embodiments, the ENaC subunit protein or mRNA (or related protein or mRNA) biomarker is on a panel of urine biomarkers tested for determining responsiveness to treatment (e.g., for hypertension). In some embodiments, provided herein are panels of two or more markers (e.g., ENaC channel subunit protein or mRNA and 1 additional marker, 2 additional markers, 5 additional markers, 10 additional markers, 20 additional markers, or more). In some embodiments, the ENaC biomarker is on a panel of urine biomarkers for identifying a variety of conditions (e.g., hypertension-related or non-hypertension related). In some embodiments, the ENaC biomarker is tested for determining the completeness of response to MR antagonist treatment. In some embodiments, the ENaC biomarker is tested for diagnosing primary or secondary aldosteronism.

### BRIEF DESCRIPTION OF THE DRAWINGS

FIG. 1 shows aldosterone binding to MR in epithelial cells of the distal nephron. The aldosterone-MR complex translocates to the nucleus, where it acts as a transcription factor for the α, β, and γ subunits of ENaC. ENaC is trafficked to the membrane, where it enhances sodium and water reabsorption and promotes potassium excretion. The aldosterone-MR complex also activates genes that prevent internalization and degradation of membrane-bound ENaC.

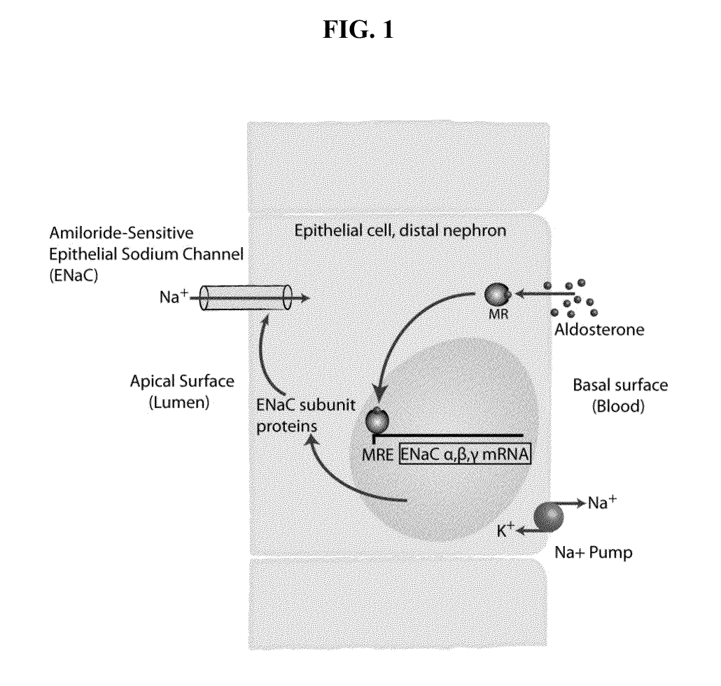

FIG. 2 shows a transmission electron micrograph of human urinary exosomes.

FIG. 3 shows a heatmap showing expression of all three ENaC subunits (SCNN1A, SCNN1B, and SCNN1G) in 11 normotensive participants' urine. In addition, 8 other mineralocorticoid receptor-regulated genes were found in human urine. Green represents lower expression (higher ΔCt), and red indicates high expression (lower ΔCt).

FIG. 4 shows an immunoblot of human sera for ENaC-α. The observed MW matches the expected MW of 75.7 kD. Serum samples were immunodepleted of the 12 most abundant serum proteins prior to immunoblotting. The two lanes for each participant represent more and less dilute samples.

FIG. 5 shows 4% agarose gel electrophoresis of PCR products from human total kidney RNA, with or without reverse transcriptase, using target-specific primers.

FIG. 6 shows 4% agarose gel electrophoresis of PCR products from human total kidney RNA, with or without reverse transcriptase, using target-specific primers.

FIG. 7 shows 4% agarose gel electrophoresis of PCR products from human total kidney RNA, with or without reverse transcriptase, using target-specific primers.

FIG. 8 shows an RT-qPCR standard curve for MR target gene SGK1 in a dilution series of renal RNA.

FIG. 9 shows RT-qPCR curves for different fragments of urinary mRNA transcripts of SCNN1A.

FIG. 10 shows RT-qPCR curves for different fragments of urinary mRNA transcripts of SCNN1B.

FIG. 11 shows RT-qPCR curves for different fragments of urinary mRNA transcripts of SCNN1G.

FIG. 12 shows RT-qPCR curves for different fragments of urinary mRNA transcripts of SKG1.

FIG. 13 shows RT-qPCR curves for different fragments of urinary mRNA transcripts of TSC22D3.

FIG. 14 shows the results of FIGS. 9-13 as a histogram.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having mineralocorticoid hypertension” refers to a subject that presents one or more signs or symptoms indicative of mineralocorticoid hypertension, has one or more risk factors for hypertension, has poor response to antihypertensive medications, is being screened for mineralocorticoid hypertension (e.g., during a routine physical), is being treated for hypertension with a mineralocorticoid receptor antagonist, or is being considered for treatment with a mineralocorticoid receptor antagonist. A subject suspected of having mineralocorticoid hypertension has generally not been tested for mineralocorticoid hypertension, or has not had a recent test which indicated the subject suffers from mineralocorticoid hypertension. However, a “subject suspected of having mineralocorticoid hypertension” encompasses an individual who has received a preliminary diagnosis but for whom a confirmatory test has not been done. A “subject suspected of having mineralocorticoid hypertension” is sometimes diagnosed with hypertension and is sometimes found to not have mineralocorticoid hypertension.

As used herein, the term “subject diagnosed with mineralocorticoid hypertension” refers to a subject who has been tested and found to have mineralocorticoid hypertension. mineralocorticoid hypertension may be diagnosed using any suitable method.

As used herein, the term “subject suffering from mineralocorticoid hypertension” refers to a subject who has mineralocorticoid hypertension and exhibits one or more signs or symptoms thereof. A subject suffering from mineralocorticoid hypertension may or may not have received a diagnosis, and may or may not be aware of the condition.

As used herein, the term “subject at risk for mineralocorticoid hypertension” refers to a subject with one or more risk factors for developing mineralocorticoid hypertension.

As used herein, the term “characterizing mineralocorticoid hypertension in subject” refers to the identification of one or more properties of mineralocorticoid hypertension in a subject (e.g. degree, severity, advancement, responsiveness to MR antagonist therapy etc.). Mineralocorticoid hypertension may be characterized by the identification of one or more markers (e.g., ENaC (e.g., in the urine (e.g., below a threshold))) described herein.

As used herein, the term “reagent(s) capable of specifically detecting biomarker expression” refers to reagents used to detect the expression of biomarkers (e.g., ENaC (e.g., in the urine (e.g., above a threshold))). Examples of suitable reagents include but are not limited to, nucleic acid probes capable of specifically hybridizing to mRNA or cDNA, and antibodies (e.g., monoclonal antibodies).

As used herein, the term “providing a prognosis” refers to providing information regarding the impact of the presence of mineralocorticoid hypertension or the responsiveness of the subject to MR antagonists on a subject's future health.

As used herein, the term “sample” is used in its broadest sense. In one sense, it is meant to include a specimen or culture obtained from any source. Biological samples may be obtained from animals (including humans) and encompass fluids, solids, tissues, and gases. Biological samples include urine, saliva, tissues, lacrimal fluid, and blood products, such as plasma, serum and the like.

As used herein, the term “antibody” is used in the broadest sense and specifically covers human, non-human (e.g. murine) and humanized monoclonal antibodies (including full length monoclonal antibodies), polyclonal antibodies, multi-specific antibodies (e.g., bispecific antibodies), single-chain antibodies, and antibody fragments so long as they exhibit the desired biological activity.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

As used herein, the term “probe” refers to an oligonucleotide (e.g., a sequence of nucleotides), whether occurring naturally as in a purified restriction digest or produced synthetically, recombinantly or by PCR amplification, that is capable of hybridizing to at least a portion of another oligonucleotide of interest (e.g., a biomarker). A probe may be single-stranded or double-stranded. Probes are useful in the detection, identification and isolation of particular gene sequences. It is contemplated that any probe used in the present invention may be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

### DETAILED DESCRIPTION

Provided herein are compositions and methods for the assessment of mineralocorticoid receptor activation or repression, and methods of customizing antihypertensive therapies based thereon. In particular, assays are provided for the detection of targets (e.g., protein, mRNA, etc.) of mineralocorticoid receptor activation (e.g., one or more of ENaC α, ENaC β, ENaC γ, GILZ, SGK1, PER1, FKBP5, RASL12, SLC12A3, TNS1, KCNJ1ENaC, etc.).

Experiments were conducted during development of embodiments described herein to quantify urinary targets (e.g., protein, mRNA, etc.) of mineralocorticoid receptor activation (e.g., one or more of GILZ, SGK1, PER1, FKBP5, RASL12, SLC12A3, TNS1, KCNJ1, and in particular epithelial sodium channel (ENaC) and subunits thereof (e.g., ENaC α, ENaC β, ENaC γ)). In some embodiments, the assays and therapies utilizing biomarkers identified in this population are provided. In certain embodiments, assays are provided that identify hypertension patients likely to respond to an MR antagonist, sparing other patients the risk of adverse effects from such treatment. In some embodiments, assays are provided to identify obesity-associated hypertension resulting from activation of the MR by an occult ligand. In some embodiments, assays are provided to identify MR activation in patients in whom aldosterone levels do not remain suppressed (e.g., approximately 30% of patients) upon taking an angiotensin-converting-enzyme (ACE) inhibitor or angiotensin-receptor blocker. In some embodiments, assays are provided to assess the adequacy of mineralocorticoid receptor antagonism in patients (e.g., heart failure patients, primary aldosteronism patients, etc.) treated with mineralocorticoid receptor antagonists.

In some embodiments, experiments conducted during development of embodiments described herein have demonstrated that centrifugation of whole human urine at less than 20,000 RPM (e.g., 1,000-14,000 RPM) for less than 1 hour (e.g., 10-20 minutes) resulted in a sample from which signal for ENaC mRNA was detected by RT-PCR. Experiments conducted during development of embodiments described herein demonstrate detection of ENaC mRNA extracted from whole urine (e.g., fresh or frozen, with or without protease inhibitors).

In some embodiments, provided herein are assays for quantitative measurement/detection/assessment, in human urine, of mRNA encoding the epithelial sodium channel (ENaC). In some embodiments, mRNA encoding one or more subunits of ENaC (e.g., α, β, and/or γ subunits) is semiquantified, or quantified in human urine. In some embodiments, assays allow researchers and/or clinicians to evaluate whether the concentration of ENaC mRNA or protein (e.g., mRNA encoding an ENaC subunit or protein for such a subunit (e.g., alpha or gamma subunit) in human urine reflects mineralocorticoid activation. Assays provided herein represent the first to measure the amount of urine ENaC mRNA or protein in humans in a state of overt excess MR activation, such as primary aldosteronism, or after resolution of excess MR activation. In some embodiments, evaluation of urinary ENaC mRNA or protein in both settings allows researchers and/or clinicians to determine whether the concentration of urinary ENaC mRNA or protein corresponds to MR activation.

In some embodiments, provided herein are indicators (e.g. biomarkers, etc.) of responsiveness or resistance to treatment (e.g., of hypertension) with MR antagonists. In some embodiments, the biomarker comprises RNA (e.g., mRNA) encoding ENaC or a subunit thereof (e.g., α-subunit, β-subunit, γ-subunit). In other embodiments, the biomarker comprises RNA (e.g., mRNA) encoding one or more of GILZ, SGK1, PER1, FKBP5, RASL12, SLC12A3, TNS1, and KCNJ1. In some embodiments, a panel of markers is identified (e.g., in urine, in blood, etc.) including one or more of SCNN1A (encoding ENaC α), SCNN1B (encoding ENaC β), SCNN1G (encoding ENaC γ), GILZ, SGK1, PER1, FKBP5, RASL12, SLC12A3, TNS1, KCNJ1 mRNA or protein and one or more (e.g., 2, 3, 4, 5, 6, 7, 8, 9, 10 . . . 20 . . . 50, etc.) additional markers (e.g., of responsiveness to MR antagonists, of hypertension, of responsiveness to other therapies, of other conditions, etc.). In some embodiments, a panel of markers comprises fewer than 10,000 markers (e.g., <10,000, <5,000, <1,000, <500, <100, <50, <20, <10). In some embodiments, ENaC mRNA or protein and/or one or more of GILZ, SGK1, PER1, FKBP5, RASL12, SLC12A3, TNS1, KCNJ1 mRNA or protein are among a panel of biomarkers for a variety of conditions (e.g., hypertension) and/or responsiveness to treatments.

In some embodiments, the kits are provided for the detection and characterization of hypertension and/or responsiveness to MR antagonists. In some embodiments, the kits contain reagents for detecting biomarkers described herein (e.g., primers, probes, and/or antibodies specific for these biomarkers), in addition to other detection reagents, amplification reagents, stabilization reagents, purification reagents, buffers, controls, etc. In certain embodiments, kits contain all of the components necessary to perform a detection assay, including all controls, directions for performing assays, and any necessary software for analysis and presentation of results. In other embodiments, one or more reagents for performing the assay are not supplied in the kit and are instead supplied by the user. In some embodiments, kits comprise instructions (e.g. written, digital, and/or online) to perform assays for the detection and characterization of hypertension and/or responsiveness to MR antagonists.

In some embodiments, methods, compositions, and systems are provided for screening arrays of compounds (e.g., pharmaceuticals, drugs, peptides, or other test compounds) for their ability to treat hypertension in subjects resistant to or responsive to MR antagonists. In some embodiments, compounds (e.g., pharmaceuticals, drugs, peptides, or other test compounds) identified using screening assays described herein find use in the diagnosis or treatment of hypertension or diagnosis of responsiveness to MR antagonists.

In some embodiments, the assays provided herein are screening assays for assessing cellular behavior or function. For example, the response of cells, tissues, or organisms to interventions (e.g., MR antagonists) may be monitored by assessing, for example, cellular functions using animal or cell culture models as described herein. Such assays find particular use for characterizing, identifying, validating, selecting, optimizing, or monitoring the effects of agents (e.g., small molecule-, peptide-, antibody-, nucleic acid-based drugs, etc.) that find use in treating or preventing hypertension or related diseases or conditions (e.g., in subject responsive to MR antagonists or resistant to treatment with MR antagonists).

Embodiments are not limited to the markers described herein. Any suitable marker that correlates with mineralocorticoid hypertension, responsiveness or resistance to MR antagonist treatment, etc., including but not limited to those described herein may find use in the embodiments described herein. Additional markers are also contemplated to be within the scope. Any suitable method may be utilized to identify and characterize markers suitable for use in the methods described herein, including but not limited to, those described herein, are within the scope described herein.

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some embodiments, data is presented that will benefit the clinician, who is not likely to be trained in molecular biology, need not understand the raw data. The data is presented directly to the clinician in a useful form. The clinician is then able to immediately utilize the information in order to optimize the care of the subject.

Provided herein are any methods capable of receiving, processing, and transmitting the information to and from laboratories conducting the assays described herein, information providers, medical personal, and subjects. For example, in some embodiments, a sample (e.g., urine sample) is obtained from a subject and submitted to a profiling service (e.g., clinical lab at a medical facility, independent testing facility, etc.), located in any part of the world (e.g., in a state or country different than where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be sent directly to the profiling service by the subject (e.g., an information card containing the information may be scanned by a computer and the data transmitted to a computer of the profiling center using an electronic communication system). Once received by the profiling service, the sample is processed and a profile is produced, specific for the diagnostic or prognostic information desired for the subject.

The profile data is then prepared in a format suitable for interpretation by a treating clinician. For example, rather than providing raw data, the prepared format may represent a diagnosis or risk assessment (e.g., likelihood of response to MR antagonist therapy) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, a risk assessment, diagnosis, prognosis, responsiveness signature, etc. is generated from an algorithm that combines multiple pieces of data (e.g., mRNA or protein levels of one or more of SCNN1A (encoding ENaC α), SCNN1B (encoding ENaC β), SCNN1 G (encoding ENaC γ), TSC22D3 (encoding GILZ), SGK1, PER1, FKBP5, RASL12, SLC12A3, TNS1, and KCNJ1), the presence of level of other downstream targets of MR, blood pressure, Na levels, etc.) to generate a result reachable only through the synergy of the multiple pieces of data, and translated via the algorithm. In some embodiments, raw data (e.g., one or more data points) is translated into predictive data by methods herein (e.g., an algorithm), and used in the field of medicine.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of the severity of disease (e.g. hypertension) or responsiveness to therapies (e.g., MR antagonists).

### EXPERIMENTAL

Experiments were conducted during development of embodiments of the technology described herein to demonstrate that downstream targets of MR activation (e.g., gene products expressed following MR activation mRNA (e.g., ENaC (or subunits thereof), etc.), etc.) is detectable in human urine (e.g., without unltracentrifugation). Consistent human urinary expression of 11 MR-regulated genes was demonstrated [SCNN1A (encoding ENaC α), SCNN1B (encoding ENaC β), SCNN1G (encoding ENaC γ), TSC22D3 (encoding GILZ), SGK1, PER1, FKBP5, RASL12, SLC12A3, TNS1, KCNJ1]. Using such detection a quantitative assay of ENaC subunit expression (mRNA and/or protein in human urine and/or sera) is developed.

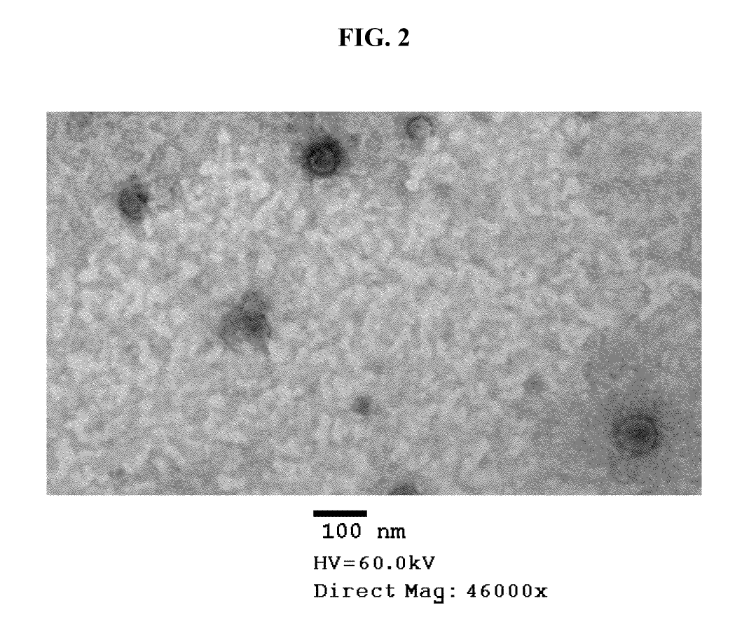

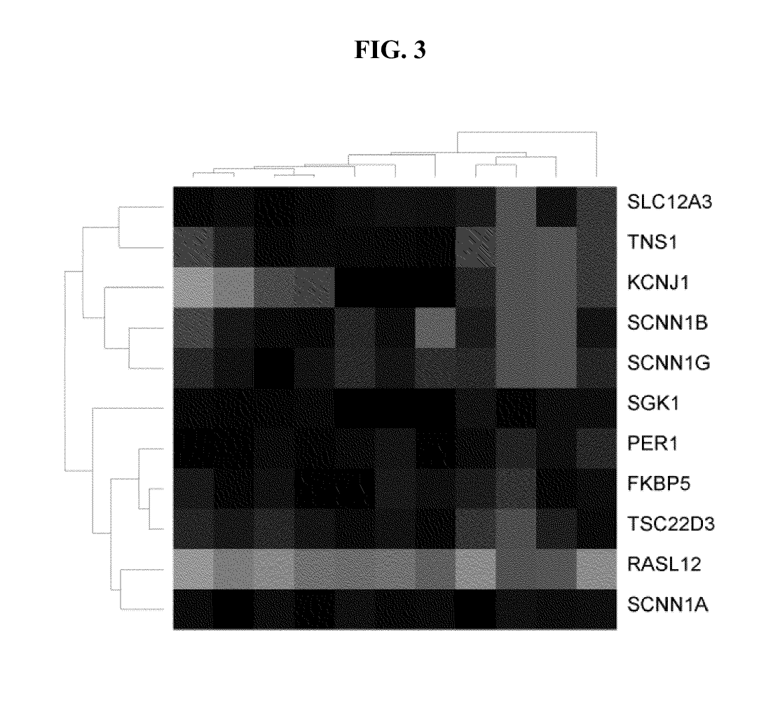

Experiments were conducted during development of embodiments described herein to isolate urinary exosomes (FIG. 2). It was then evaluated whether ENaC subunit expression in human urinary exosomes and/or human urine could be detected with the cellular elements intact. mRNA was isolated from urinary exosomes or from whole urine (with the cellular element preserved). Using either of these starting materials, all three ENaC subunits were detected using ABI TaqMan probes (FIG. 3). In addition, 8 other genes known to be regulated by MR in animal models were detected (FIG. 3). The methods used to isolate exosomal mRNA or total mRNA can be replicated in a clinical laboratory (e.g., no ultracentrifugation is required). The mRNA for all three ENaC subunits was detected in exosomes or exfoliated cells isolated from human urine. ENaC subunit mRNA has been detected in human urine irrespective of whether a preservative (e.g., Norgen, catalogue number 18122) is added. It is contemplated that the exosomes provide a sheltering environment for mRNA. No-template controls were negative for expression of all genes at 40 cycles, and human adrenal cells (HAC-15 cells) were positive, as would be expected. The ability to detect ENaC subunit mRNA in urine stored for days (e.g., 1 day, 2 days, 3 days, 4 days, 5 days, etc.) without ultracentrifugation or special collection methods indicates that this approach is suitable for a clinically useful assay.

The urine of normotensive individuals was assayed to determine whether urinary mRNA for ENaC subunits is detectable at a consistent GAPDH-normalized cycle number. Consistent urinary expression of all three ENaC subunits' mRNA was detected in normotensive humans.

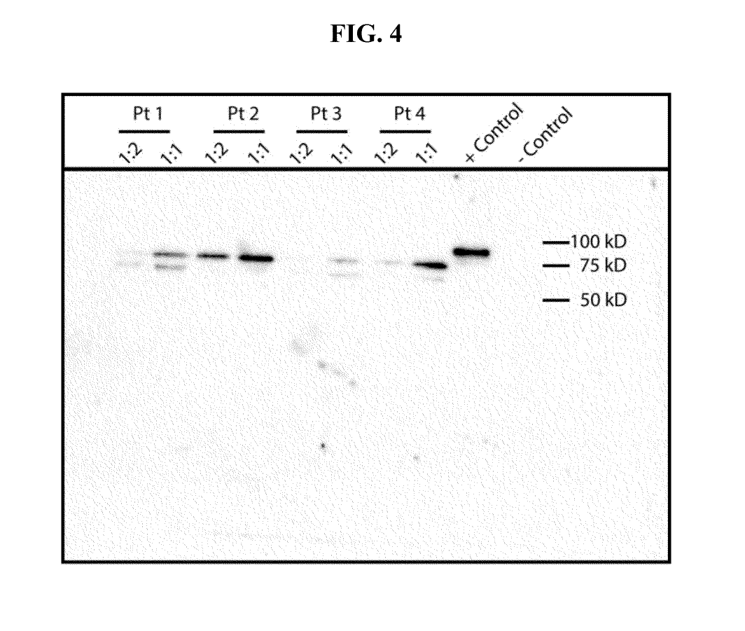

ENaC is expressed on isolated human peripheral blood mononuclear cells, and its expression on these cells is decreased in patients treated with spironolactone. In order evaluate whether ENaC is detectable in human sera, immunoblotting was performed on sera from human subjects (FIG. 4).

In some embodiments, detection and/or quantification of biomarkers and/or analytes in a sample (e.g., urine) comprises target-specific amplification of mRNA in the sample prior to quantitative PCR and/or other detection quantification steps. Table 1 described exemplary primer and probe sequences (or binding sequences in target analytes) for the detection of exemplary target sequences.

TABLE 1

| | | Position relative | |
|---|---|---|---|
| | | to the first | |
| SEQ | | base of the 5′ | |
| ID | | untranslated | |
| NO. | Target | regions | sequence |
| 1 | SCNN1B | T 456 . . . 475 | CAATGCTAGCCCCTTCAAGT |
| 2 | SCNN1B | B 513 . . . 531 | CAGGACAGCTTCCATCAGC |
| 3 | SCNN1B | T 1021 . . . 1039 | GCCAACCCTGGAACTGAAT |
| 4 | SCNN1B | B 1117 . . . 1136 | GACCTCTGCTCGTGAAGCAT |
| 5 | SCNN1B | T 1608 . . . 1627 | CACCAATATCACCCTGAGCA |
| 6 | SCNN1B | B 1708 . . . 1727 | AGATTCGAGAGCAGCCAGAC |
| 7 | SCNN1B | T 1577 . . . 1596 | TCCACGTCTTGTCTCAGGAG |
| 8 | SCNN1B | B 1735 . . . 1754 | CCCATCCAGAAGCCAAACTG |
| 9 | SCNN1B | T 983 . . . 1002 | TCTTCAACTGGGGCATGACA |
| 10 | SCNN1B | B 1176 . . . 1194 | GATGGACGTCTCTGTCCCC |
| 11 | SCNN1B | T 417 . . . 436 | AGGCTTCAAGACCATGGACT |
| 12 | SCNN1B | B 549 . . . 568 | CATTGGCATGGCTTAGCTCA |
| 13 | TSC22D3 | B 720 . . . 739 | ATGGCCTGTTCGATCTTGTT |
| 14 | TSC22D3 | T 667 . . . 686 | CCATCCTGCTCTTCTTCCAC |
| 15 | TSC22D3 | T 725 . . . 743 | GATCGAACAGGCCATGGAT |
| 16 | TSC22D3 | B 807 . . . 825 | TCTTCTCCACCAGCTCTCG |
| 17 | TSC22D3 | T 607 . . . 626 | ACCCCTGCTACCTGATCAAC |
| 18 | TSC22D3 | B 770 . . . 789 | TCTCCACCTCCTCTCTCACA |
| 19 | SGK1 | T 817 . . . 836 | TTTCCAAAGAGGGGTTCTCC |
| 20 | SGK1 | B 889 . . . 908 | TGGCATGATTACATGGCTCT |
| 21 | SGK1 | B 1145 . . . 1164 | TTAGCATGAGGATTGGACGA |
| 22 | SGK1 | T 1072 . . . 1092 | TCAGGAGCCTGAGCTTATGAA |
| 23 | SGK1 | T 1706 . . . 1725 | GACAGGACTGTGGACTGGTG |
| 24 | SGK1 | B 1777 . . . 1796 | TTTCAGCTGTGTTTCGGCTA |
| 25 | SGK1 | T 716 . . . 735 | AGTCCCAGCCTGAAGTACAC |
| 26 | SGK1 | B 920 . . . 939 | AAGGTTCTTGGATCGGGCTT |
| 27 | SGK1 | T 1025 . . . 1044 | GCATGCAAACACCCTGAAGT |
| 28 | SGK1 | B 1195 . . . 1214 | TTCCAAAACTGCCCTTTCCG |
| 29 | SGK1 | T 1632 . . . 1651 | ACAACAGCACAACATCCACC |
| 30 | SGK1 | B 1847 . . . 1866 | AGGAGGTGTCTTGCGGAATT |

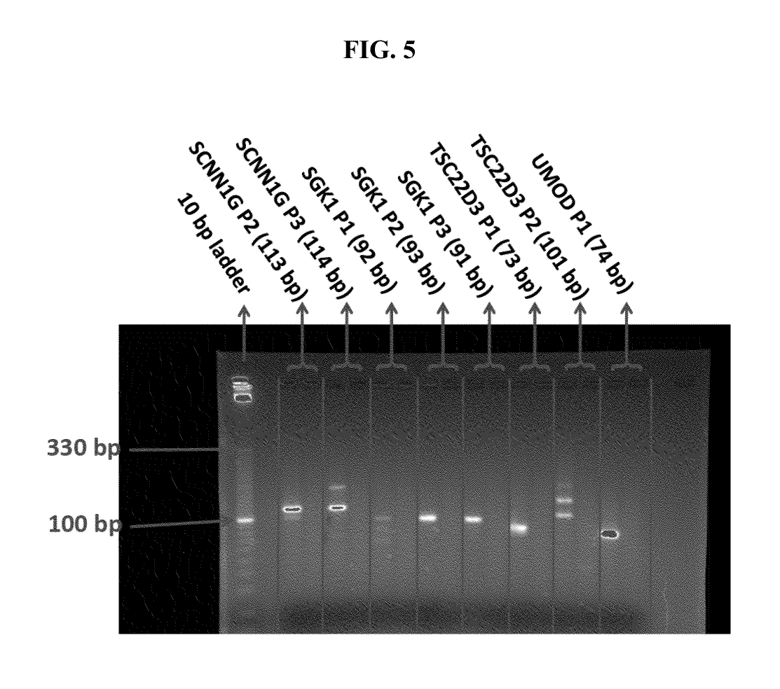

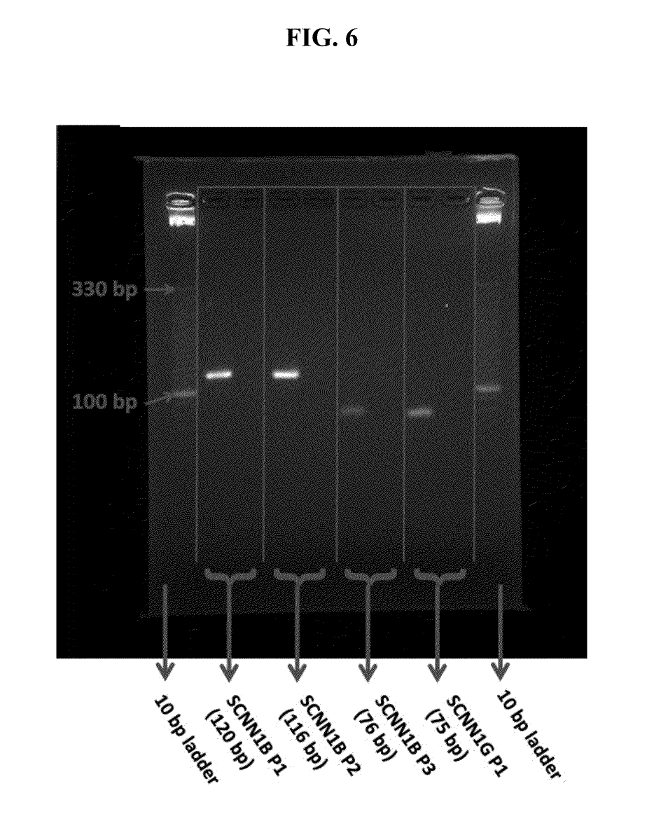

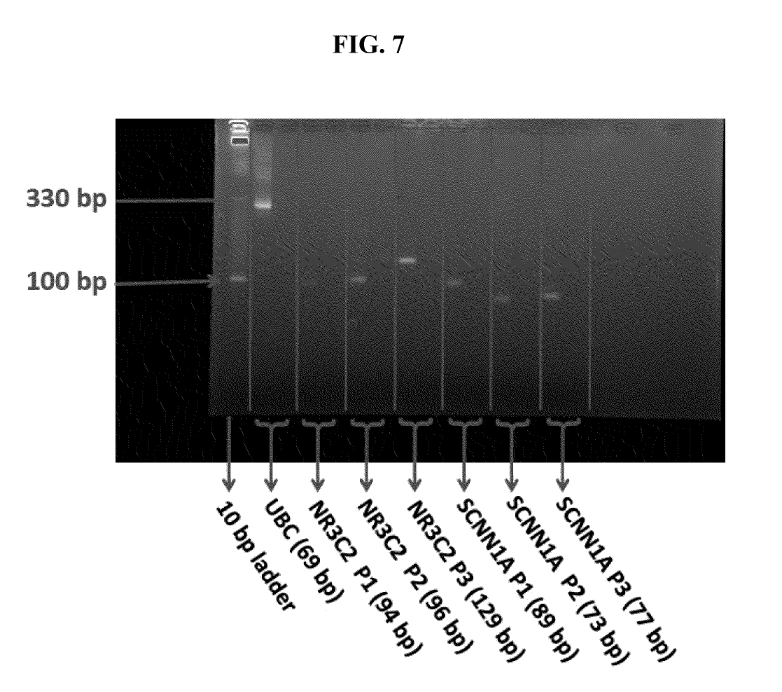

Alternative primer and probe sequences and/or variations of the exemplary sequences above (e.g., >50 sequence identity (e.g., >55%, >60%, >65%, >70%, >75%, >80%, >90%, >95%) are within the scope herein. FIGS. 5-7 depict 4% agarose gel electrophoresis of PCR products, with or without reverse transcriptase, using, for example, primers of Table 1. DNA was visualized using SYBR Safe stain. RNA template was human total kidney RNA. These experiments demonstrate detection of the targets in a positive control (human total kidney RNA).

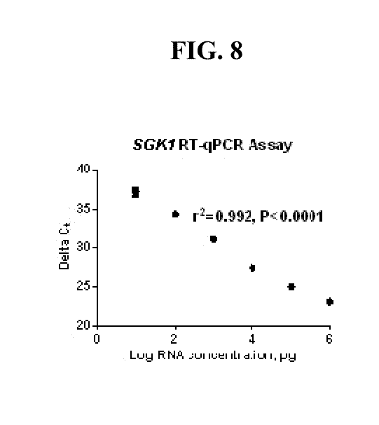

Experiments were conducted during development of embodiments herein in which RT-qPCR standard curves were produced for MR target genes in a dilution series of renal RNA. As depicted in FIG. 8 for the MR target gene SGK1, the RT-qPCR assay has exceptional sensitivity, as well as linearity across at least 6 logs of RNA concentration.

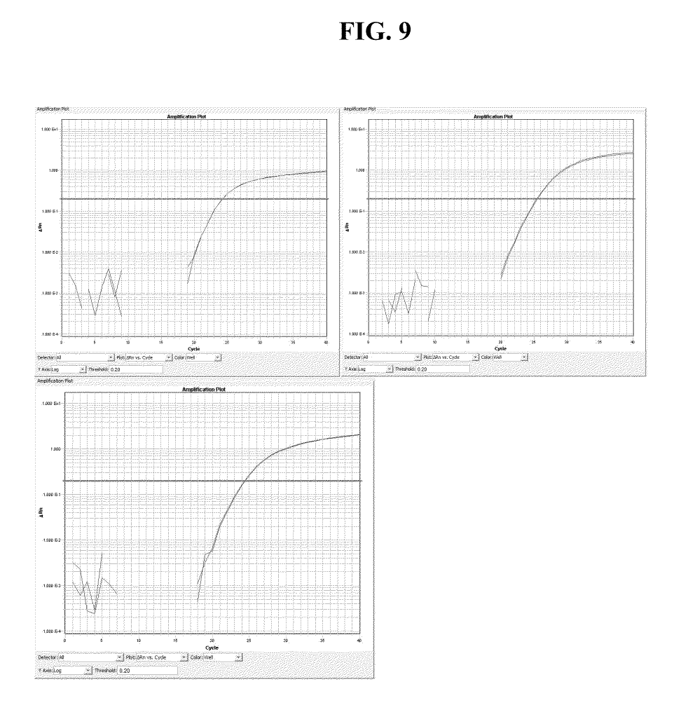

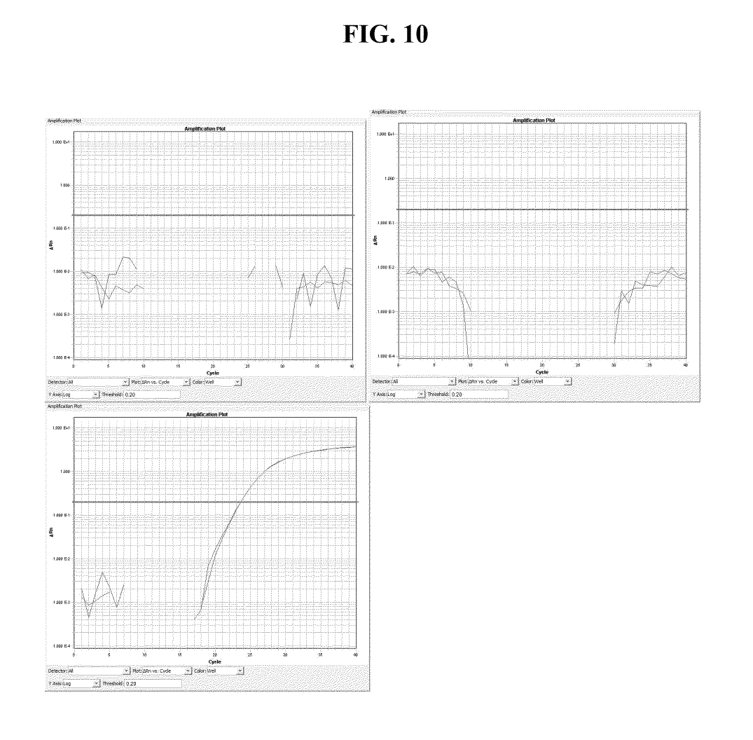

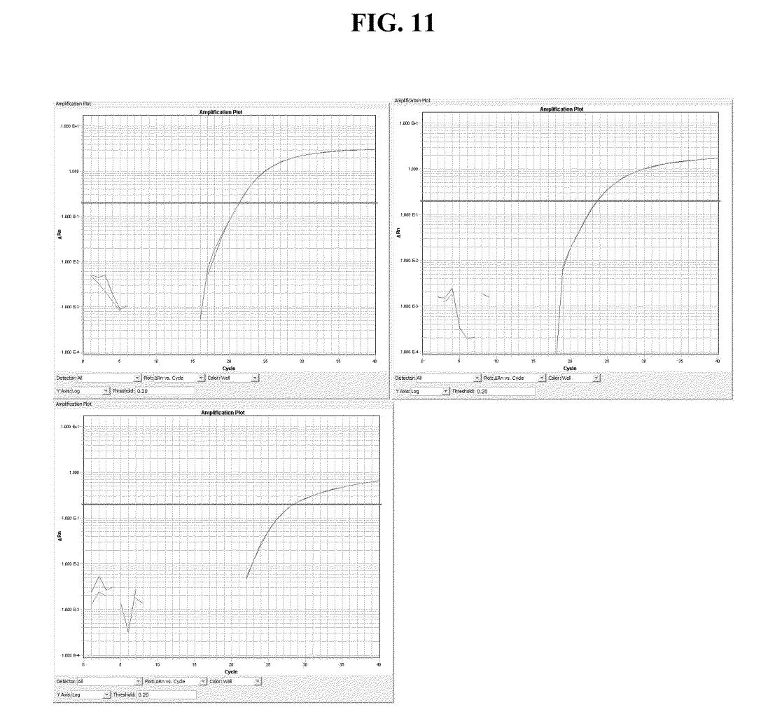

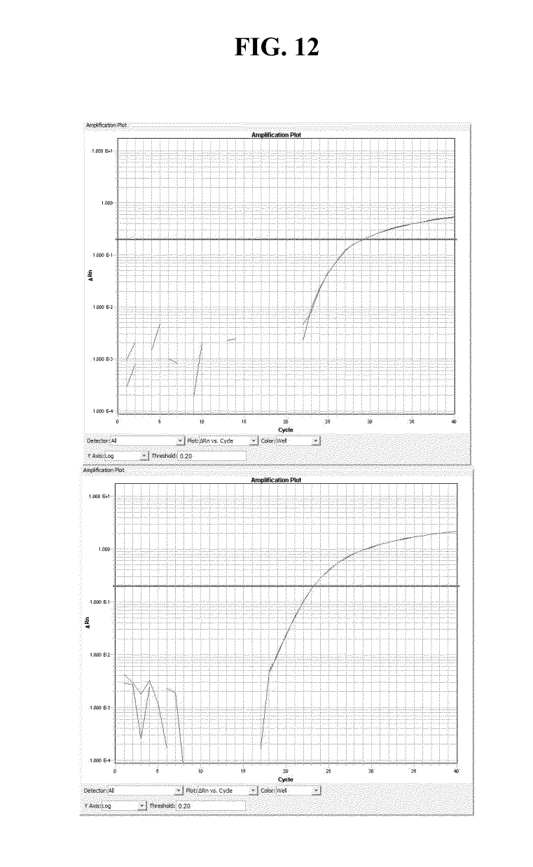

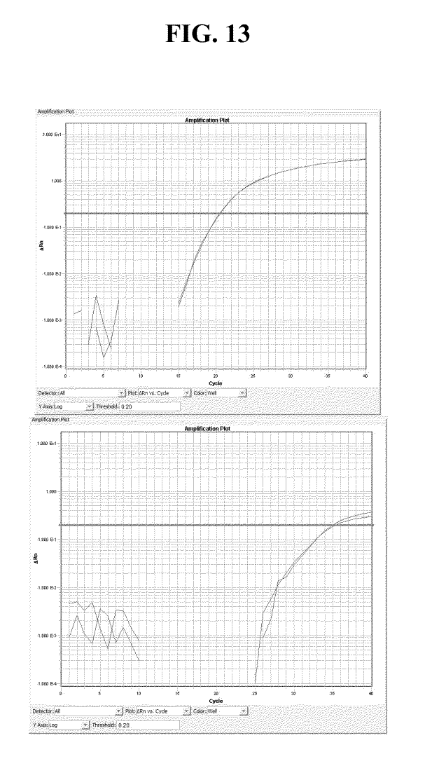

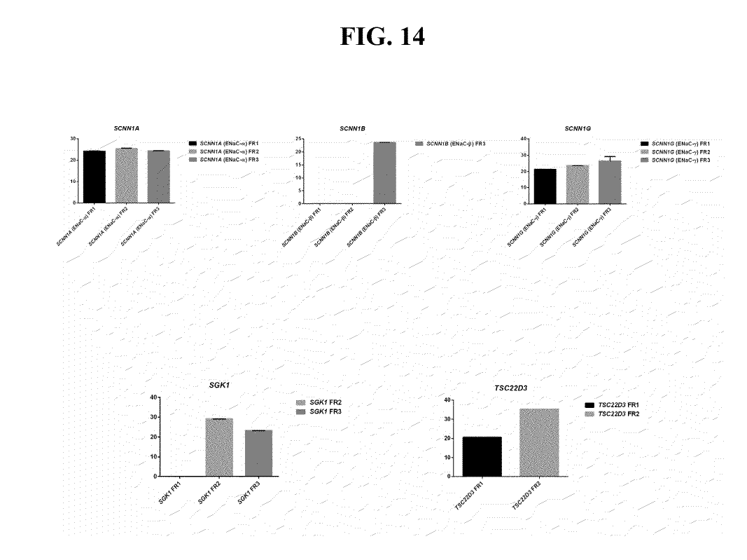

In some embodiments, due to fragmentation of mRNA in biological samples (e.g., urine samples), it is not possible to detect analytes using primers or probes for any portion of the analyte sequence. In some embodiments, target-specific primers and probes are designed (e.g., See Table 1) that detect portions of the analyte that survive and/or are detectable following fragmentation. In some embodiments, amplification/detection/quantification is carried our using primers and probes for multiple regions along an analyte to increase the likelihood (e.g., to ensure) detection of the analyte if present (e.g., even if fragmented). In some embodiments, different fragments of an mRNA transcript of a target analyte are quantified at different levels (See, e.g., FIGS. 9-14). Based upon results of experiments conducted during development of embodiments herein, in some embodiments, multiple primers sets and/or probes are utilized in an assay to increase coverage of the target gene. For example, FIG. 10 demonstrates that SCNN1B FR1 was not detectable while SCNN1B FR3 was. Using primers and probes for the detection of both fragments enables detection of the analyte in the urine sample, whereas use of reagents for the detection of SCNN1B FR1 alone would not.

Experiments were conducted during development of embodiments herein demonstrate detection/quantification of the analytes described herein (e.g., target of MR activation) in urine samples that have not been subjected to ultracentrifugal forces.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

### REFERENCES

The following references are herein incorporated by reference in their entireties.
Carter et al. Am J Physiol Cell Physiol. 2001 November; 281(5):C1413-21.
Lauridsen, BMC Nephrology 2010, 11:28.
Edwards & Korchev. WO2007042776.
JAISSER et al. WO/2010/046411.
Narikiyo et al. J Clin Invest. 2002 February; 109(3):401-8.
Lienhard et al. PLoS ONE 7(5): e37898. May 31, 2012.
West et al. Am J Physiol Regul Integr Comp Physiol. 2010 November; 299(5): R1326-R1332.
Danziger & Skidgel, US20080026409A1.
AKINS et al., WO2004006844A2.
Klass et al. US20100184046A1.
SKOG, US20100196426A1.
Toshimitsu Niwa, Journal of Chromatography B Volume 870, Issue 2, 15 Jul. 2008, Pages 148-153
Hoorn et al. Nephrology (Carlton), 2005 June; 10(3):283-90.
Lauridsen, et al. European Journal of Endocrinology (2010) 162 961-969.
Eudy et al. Journal of Translational Medicine 2011, 9:180.
Olivieri et al. Hypertension. 2005; 46:683-688.
Meiracker et al. Hypertension. 2012; 60:741-748.
Koda et al. Hypertension Research (2009) 32, 276-281.
Matthesen et al. Clinical and Experimental Hypertension, 2012; Early Online: 1-13.
Esteva-Nephron Physiol 2010; 114:p 25-p 34.
Zhu et al. Pediatric Research 65: 443-446, 2009.
Pisitkun et al. 13368-13373 PNAS Sep. 7, 2004 vol. 101 no. 36
Graffe et al. BMC Nephrology 2012, 13:15.
Graffe et al. Am J Physiol Renal Physiol 302: F917-F927, 2012.
Lauridsen et al. European Journal of Endocrinology (2010) 162 961-969.
Lauridsen et al. Nephrol Dial Transplant (2010) 25: 2502-2510.
Rennings et al. Clinical pharmacology & Therapeutics |VOLUME 89 NUMBER 4|April 2011

1. A method for detecting one or more target analytes that are indicative of mineralocorticoid receptor activation in a sample, the method comprising exposing a urine sample to detection reagents that are specific for the target analytes, wherein the urine sample has not been subjected to ultracentrifugation.

2. The method of claim 1, wherein the target analytes are selected from SCNN1A (encoding ENaC α), SCNN1B (encoding ENaC β), SCNN1G (encoding ENaC γ), TSC22D3 (encoding GILZ), SGK1, PER1, FKBP5, RASL12, SLC12A3, TNS1, and KCNJ1.

3. The method of claim 1, wherein the target analytes are selected from Akap12, Ophn1, Apbb3, Per1, Asap1, Cp, Ctgf, Slc45a1, Fgd3, Slco3a1, Synpo, Ikzf4, Tgfa, Klf6, Klf9, Mrpl33, Tspan2, Msi2, Zfand5, and Ngf.

4. The method of claim 1, wherein the urine sample is processed, but not ultracentrifuged.

5. The method of claim 1, wherein the target analytes are mRNA transcripts of genes expressed following mineralocorticoid receptor activation, or nucleic acid fragments thereof.

6. The method of claim 5, wherein the detection reagents comprise detectably-labeled nucleic acid probes that specifically hybridize to the target analytes or amplification products thereof.

7. The method of claim 6, wherein the detection reagents are fluorescently labeled.

8. The method of claim 7, wherein detection reagents are selected from (i) non-specific fluorescent dyes that intercalate amplification products of target analytes, and (ii) fluorescently-labeled and target-specific oligonucleotide probes.

9. The method of claim 1, further comprising exposing a urine sample to amplification reagents that are specific for the target analytes.

10. The method of claim 1, wherein the amplification reagents comprise target-analyte-specific primers.

11. The method of claim 10, wherein the urine sample is exposed to two or more pairs of target-analyte-specific primers for each target analyte.

12. The method of claim 11, wherein the urine sample is contacted with a detection reagent for each of the two or more pairs of target-analyte-specific primers.

13. The method of claim 1, comprising the steps of
(a) obtaining or receiving the urine sample;
(b) processing the urine sample;
(c) amplifying portions of one or more of the target analytes using target-analyte-specific primers pairs to produce one or more target-analyte-specific amplicons;
(d) contacting the urine sample with at least one detection probe for each of the one or more target-analyte-specific amplicons.


SEQUENCE LISTING (30 sequences)

SEQ ID NO: 1 - DNA, 20 bp, Artificial sequence
  DNA primer/probe
caatgctagccccttcaagt

SEQ ID NO: 2 - DNA, 19 bp, Artificial sequence
  DNA primer/probe
caggacagcttccatcagc

SEQ ID NO: 3 - DNA, 19 bp, Artificial sequence
  DNA primer/probe
gccaaccctggaactgaat

SEQ ID NO: 4 - DNA, 20 bp, Artificial sequence
  DNA primer/probe
gacctctgctcgtgaagcat

SEQ ID NO: 5 - DNA, 20 bp, Artificial sequence
  DNA primer/probe
caccaatatcaccctgagca

SEQ ID NO: 6 - DNA, 20 bp, Artificial sequence
  DNA primer/probe
agattcgagagcagccagac

SEQ ID NO: 7 - DNA, 20 bp, Artificial sequence
  DNA primer/probe
tccacgtcttgtctcaggag

SEQ ID NO: 8 - DNA, 20 bp, Artificial sequence
  DNA primer/probe
cccatccagaagccaaactg

SEQ ID NO: 9 - DNA, 20 bp, Artificial sequence
  DNA primer/probe
tcttcaactggggcatgaca

SEQ ID NO: 10 - DNA, 19 bp, Artificial sequence
  DNA primer/probe
gatggacgtctctgtcccc

SEQ ID NO: 11 - DNA, 20 bp, Artificial sequence
  DNA primer/probe
aggcttcaagaccatggact

SEQ ID NO: 12 - DNA, 20 bp, Artificial sequence
  DNA primer/probe
cattggcatggcttagctca

SEQ ID NO: 13 - DNA, 20 bp, Artificial sequence
  DNA primer/probe
atggcctgttcgatcttgtt

SEQ ID NO: 14 - DNA, 20 bp, Artificial sequence
  DNA primer/probe
ccatcctgctcttcttccac

SEQ ID NO: 15 - DNA, 19 bp, Artificial sequence
  DNA primer/probe
gatcgaacaggccatggat

SEQ ID NO: 16 - DNA, 19 bp, Artificial sequence
  DNA primer/probe
tcttctccaccagctctcg

SEQ ID NO: 17 - DNA, 20 bp, Artificial sequence
  DNA primer/probe
acccctgctacctgatcaac

SEQ ID NO: 18 - DNA, 20 bp, Artificial sequence
  DNA primer/probe
tctccacctcctctctcaca

SEQ ID NO: 19 - DNA, 20 bp, Artificial sequence
  DNA primer/probe
tttccaaagaggggttctcc

SEQ ID NO: 20 - DNA, 20 bp, Artificial sequence
  DNA primer/probe
tggcatgattacatggctct

SEQ ID NO: 21 - DNA, 20 bp, Artificial sequence
  DNA primer/probe
ttagcatgaggattggacga

SEQ ID NO: 22 - DNA, 21 bp, Artificial sequence
  DNA primer/probe
tcaggagcctgagcttatgaa

SEQ ID NO: 23 - DNA, 20 bp, Artificial sequence
  DNA primer/probe
gacaggactgtggactggtg

SEQ ID NO: 24 - DNA, 20 bp, Artificial sequence
  DNA primer/probe
tttcagctgtgtttcggcta

SEQ ID NO: 25 - DNA, 20 bp, Artificial sequence
  DNA primer/probe
agtcccagcctgaagtacac

SEQ ID NO: 26 - DNA, 20 bp, Artificial sequence
  DNA primer/probe
aaggttcttggatcgggctt

SEQ ID NO: 27 - DNA, 20 bp, Artificial sequence
  DNA primer/probe
gcatgcaaacaccctgaagt

SEQ ID NO: 28 - DNA, 20 bp, Artificial sequence
  DNA primer/probe
ttccaaaactgccctttccg

SEQ ID NO: 29 - DNA, 20 bp, Artificial sequence
  DNA primer/probe
acaacagcacaacatccacc

SEQ ID NO: 30 - DNA, 20 bp, Artificial sequence
  DNA primer/probe
aggaggtgtcttgcggaatt